(19) United States
(12) Patent Application Publication
(10) Pub. No.: US 2013/0184728 A1
(43) Pub. Date: Jul. 18, 2013

(54) Ultrasound Neuromodulation for Diagnosis and Other-Modality Preplanning

(71) Applicant: David J. Mishelevich, Playa del Rey, CA (US)

(72) Inventor: David J. MISHELEVICH, Playa del Rey, CA (US)

(73) Assignee: David J. MISHELEVICH, Playa del Rey, CA (US)

(21) Appl. No.: 13/718,245

(22) Filed: Dec. 18, 2012

Related U.S. Application Data
(63) Continuation-in-part of application No. 13/689,178, filed on Nov. 29, 2012.
(60) Provisional application No. 61/564,856, filed on Nov. 29, 2011.
(60) Provisional application No. 61/577,095, filed on Dec. 19, 2011.

(51) Int. Cl. A61N 7/02

(52) U.S. Cl. CPC A61N 7/02
USPC 606/169

(57) ABSTRACT

Disclosed are methods and systems for non-invasive neuromodulation using ultrasound for diagnosis to evaluate the feasibility of and preplan neuromodulation treatment using other modalities. The neuromodulation can produce acute or long-term effects. The latter occur through Long-Term Depression (LTD) and Long-Term Potentiation (LTP) via training Included is control of direction of the energy emission, intensity, frequency, pulse duration, pulse pattern, mechanical perturbation, and phase/intensity relationships to targeting and accomplishing up regulation and/or down regulation.

### CROSS-REFERENCE

This application is a continuation-in-part application of U.S. patent application Ser. No. 13/689,178, filed Nov. 29, 2012, entitled “ULTRASOUND NEUROMODULATION OF SPINAL CORD” (attorney docket no. 42927-716.201); which application claims priority to U.S. Provisional Application Ser. No. 61/564,856, filed Nov. 29, 2011, entitled “ULTRASOUND NEUROMODULATION OF SPINAL CORD” (attorney docket no. 42927-716.101); and claims priority to U.S. Application Ser. No. 61/577,095, “ULTRASOUND NEUROMODULATION FOR DIAGNOSIS AND OTHER-MODALITY PREPLANNING,” filed Dec. 18, 2011 (attorney docket no. 42927-717.101); the full disclosures of which are incorporated herein by reference in their entirety and to which priority is claimed under 35 U.S.C. §§120 and 119.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

The present invention relates generally to systems and methods for neuromodulation and more particularly to systems and methods for diagnosis and treatment with ultrasound.

### BACKGROUND OF THE INVENTION

Although it has been demonstrated that focused ultrasound directed at neural structures can stimulate those structures, the prior methods and apparatus have lead to less than ideal results in at least some instances.

If neural activity is increased or excited, the neural structure is up regulated; if neural activated is decreased or inhibited, the neural structure is down regulated. Neural structures are usually assembled in circuits. For example, nuclei and tracts connecting them make up a circuit.

The effect of ultrasound on neural activity appears to be at least two fold. Firstly, increasing temperature will increase neural activity. Secondly, mechanical perturbation appears to be related to the opening of ion channels related to neural activity.

With regards to increasing temperature, an increase up to 42 degrees C. (say in the range of 39 to 42 degrees C.) locally for short time periods will increase neural activity in a way that one can do so repeatedly and be safe. For clinical uses, one needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel.

With regards to mechanical perturbation, an explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)), in which publication voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. Tyler incorporated this approach in two patent applications he submitted (Tyler, William, James P., PCT/US2009/050560, WO 2010/009141, “Methods and Devices for Modulating Cellular Activity Using Ultrasound,” published 2011 Jan. 21 and “Devices and Methods for Modulating Brain Activity,” PCT/US2010/055527, WO 2011/057028, published 2011 May 12). Alternative mechanisms for the effects of ultrasound may be discovered as well. In fact, multiple mechanisms may come into play.

Approaches to date of delivering focused ultrasound vary, and the clinical results and predictability can be less than ideal in at least some instances. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. The position of focus may be obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits.

Deisseroth and Schneider (U.S. patent application Ser. No. 12/263,026 published as US 2009/0112133 A1, Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts.

Many patients suffer from diseases and conditions that may be less than ideally treated. For example, patient conditions having similar symptoms can make it difficult to determine the underlying cause of the patient's symptoms. Also, at least some therapies may provide less than ideal results in at least some instances, and it would be helpful to use presently available therapies more effectively.

Because of the utility of ultrasound in the neuromodulation of neurological structures such as deep-brain structures, it would be both beneficial and desirable to provide improved diagnosis of patient conditions and improved treatment planning

### SUMMARY OF THE INVENTION

The embodiments described herein provide improved methods and systems for patient diagnosis or patient treatment planning The systems and methods may provide non-invasive neuromodulation using ultrasound for diagnosis or treatment of the patient. The systems and methods can be well suited for diagnosing one or more conditions of the patient from among a plurality of possible conditions having one or more similar symptoms. The treatment planning may comprise pre-treatment planning based on ultrasonic assessment with focused ultrasonic pulses directed to one or more target locations of the patient. Based on the evaluation of symptoms or other outcomes in response to targeting a location with ultrasound, the patient treatment at the target location can be confirmed before the patient is treated.

In a first aspect, embodiments provide a method of neuromodulation of a patient. A pulsed ultrasound is provided to one or more neural targets. A neural disorder is identified or treatment is planned for the neural disorder based on a response of the one or more neural targets to the pulsed ultrasound.

In another aspect, embodiments provide a system for neuromodulation. The system comprises circuitry coupled to one or more ultrasound transducers to provide pulsed ultrasound to one or more neural targets. A processor is coupled to the circuitry. The processor is configured to identify a neural disorder or plan for treatment of the neural disorder based on a response of the one or more neural targets to the pulsed ultrasound.

The ultrasound pulses as described herein can be used in many ways. The pulses can be used at one or more sessions to diagnose the patient, confirm subsequent treatment, or treat the patient, and combinations thereof. The pulses can be shaped in one or more ways, and can be shaped with macro pulse shaping, amplitude modulation of the pulses, and combinations thereof, for example.

In many embodiments, the amplitude modulation frequency of lower than 500 Hz is applied for inhibition of neural activity. The amplitude modulation frequency of lower than 500 Hz can be divided into pulses 0.1 to 20 msec. repeated at frequencies of 2 Hz or lower for down regulation. The amplitude modulation frequency for excitation can be in the range of 500 Hz to 5 MHz. The amplitude modulation frequency of 500 Hz or higher may be divided into pulses 0.1 to 20 msec. repeated at frequencies higher than 2 Hz for up regulation.

In many embodiments, the spinal cord can be treated. Target regions in the spinal cord which can be treated using the ultrasound neuromodulation protocols of the present invention comprise the same locations targeted by electrical SCS electrodes for the same conditions being treated, e.g., a lower cervical-upper thoracic target region for angina, a T5-7 target region for abdominal/visceral pain, and a T10 target region for sciatic pain. Ultrasound neuromodulation in accordance with the present invention can stimulate pain inhibition pathways that in turn can produce acute and/or long-term effects. Other clinical applications of ultrasound neuromodulation of the spinal cord include non-invasive assessment of neuromodulation at a particular target region in a patient's spinal cord prior to implanting an electrode for electrical spinal cord stimulation for pain or other conditions.

In many embodiments the ultrasound neuromodulation of the target may include non-invasive assessment of neuromodulation at a particular target neural region in a patient prior to implanting an electrode for electrical stimulation for pain or other conditions as described herein.

In many embodiments, the feasibility of using Deep Brain Stimulation (DBS) is determined for treatment of depression and to test whether depression symptoms can be mitigated with stimulation of the Cingulate Genu. Dramatic results may occur in some patients (e.g., description as having “lifted the void”). Such results, however, may not occur, so neuromodulation of the Cingulate Genu with ultrasound and determining the patient's response can identify those who would benefit from DBS of that target so as to confirm treatment of the Cingulate Genu target.

In many embodiments, the target site for DBS for the treatment of motor symptoms (e.g., bradykinesia, stiffness, tremor) of Parkinson's Disease (PD) comprises the Subthalamic Nucleus (STN). Stimulation of the STN may well have side effects (e.g., problems with speech, swallowing, weakness, cramping, double vision) because sensitive structures are close to it. An alternative target for the treatment of Parkinson's Disease is the Globus Pallidus interna (GPi) which can be effective in motor symptoms as well as dystonia (e.g., posturing and painful cramping). Which of these two targets will overall be best for a given patient depends on that patient and can be determined based on the patient response to DBS. Stimulation of either the GPi or STN improves many features of advanced PD, and even though STN stimulation can be effective, stimulation of the GPi can be an appropriate DBS target to determine whether the STN or GPi should be treated.

In many embodiments, the target comprises the Ventral Intermediate Nucleus of the Thalamus (Vim), which is related to motor symptoms such as essential tremor. In some embodiments, patients with tremor as their dominant symptom benefit from Vim stimulation even though other symptoms are not ameliorated, since such stimulation can deliver the best “motor result.”

In many embodiments, DBS is used on both the STN and the Vim on the same side, such that a plurality of target sites is confirmed and treated.

In many embodiments, ultrasound neuromodulation is used to select the best target for the given patient with the given condition based on testing the results of stimulating different targets. DBS stimulation of each of the potential Parkinson's Disease targets may elicit side effects that are patient specific, for example targets comprising one or more of STN, GPi, or Vim. Alternatively or in combination, ultrasound neuromodulation of the spinal cord can be used to assess whether pain has been relieved and to evaluate the potential effectiveness of or parameters for Spinal Cord Stimulation (SCS) using invasive electrode stimulation.

In many embodiments related to diagnosis and preplanning, patient feedback can be used to adjust ultrasound neuromodulation parameters for at least some conditions as described herein. In some embodiments, ultrasound neuromodulation can be used to retrain neural pathways over time, such that the patient can be treated without constant stimulation of DBS.

Alternatively or in combination with preplanning, ultrasound neuromodulation can be used to diagnosis the patient. In many embodiments, an accurate diagnosis may be difficult with prior methods and apparatus because of the way the disorder manifests itself. In many embodiments, diagnostic the methods and apparatus as described herein provide differentiation between the tremor of Parkinson's Disease and essential tremor. In many embodiments, the tremor of Parkinson's Disease typically occurs at rest and essential tremor does not or is accentuated by movement. An area of confusion is that some patients with Parkinson's Disease have tremor at rest as well.

The methods and apparatus as described herein provide a higher probability of getting the correct diagnosis and can differentiate between essential tremor and the tremor of Parkinson's Disease, such that the patient can be provided with proper treatment. The drug treatments are different for Parkinson's disease and essential tremor. The treatment of Parkinson's Disease in accordance with embodiments comprises treatment with one or more of levodopa, dopamine agonists, MAO-B inhibitors, and other drugs such as amantadine and anticholinergics. The treatment of essential tremor comprises one or more of beta blockers, propranolol, antiepileptic agents, primidone, or gabapentin. The higher probability of getting the right diagnosis can be beneficial with respect to drug treatment in a number of people with essential tremor who may also suffer fear of public situations. In at least some embodiments, medicines used to treat essential tremor may also increase a person's risk of becoming depressed. Embodiments as described herein can improve surgical treatments, as pallidotomy or thalamotomy can be used for either Parkinson's Disease or essential tremor but pallidotomy is generally not effective for essential tremor. The diagnostic methods and apparatus can differentiate between Parkinson's disease and essential tremor, for example when imaging by one or more of CT or MRI scans is insufficient to make a diagnosis. Many embodiments provide the ability to allow the correct selection of therapies selected from among one or more of surgical, neuromodulation, or drug therapies.

While ultrasound neuromodulation can produce acute effects or Long-Term Potentiation (LTP) or Long-Term Depression (LTD), the acute effects are used in many embodiments as described herein. The embodiments as described herein provide control of direction of the energy emission, intensity, frequency (carrier frequency and/or neuromodulation frequency), pulse duration, pulse pattern, and phase/intensity relationships to targeting and accomplishing up-regulation and/or down-regulation. Ancillary monitoring or imaging to provide feedback can be optionally and beneficially combined with the ultrasonic systems and methods as described herein. In many embodiments where concurrent imaging is performed, such as MRI imaging, the systems and methods may comprise non-ferrous material.

In many embodiments, single or multiple targets in groups can be neuromodulated to evaluate the feasibility of treatment and to preplan treatment using neuromodulation modalities, which may comprise non-ultrasonic or ultrasonic modalities, for example. To accomplish this evaluation, in some embodiments the neural targets will be up regulated and in some embodiments down regulated, and combinations thereof, depending on the identified neural target under evaluation. In many embodiments, the targets can be identified by one or more of PET imaging, fMRI imaging, clinical response to Deep-Brain Stimulation (DBS), or Transcranial Magnetic Stimulation (TMS).

In many embodiments, the identified targets depend on the patient and the relationships among the targets of the patient. In some embodiments, multiple neuromodulation targets will be bilateral and in other embodiments ipsilateral or contralateral. The specific targets identified and/or whether the given target is up regulated or down regulated, can depend upon the individual patient and the relationships of up regulation and down regulation among targets, and the patterns of stimulation applied to the targets identified for the patient.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and in terms of the cost of administering the therapy.

While ultrasound can be focused down to a diameter on the order of one to a few millimeters (depending on the frequency), whether such a tight focus is required depends on the configuration of the neural target. In order to determine feasibility or preplan treatment by an invasive neuromodulation modality a non-invasive mechanism must be used. Among non-invasive methods, ultrasound neuromodulation is more focused than Transcranial Magnetic Stimulation so it inherently offers more capability to demonstrate the feasibility of and preplan treatment planning for invasive and in many cases highly focused neuromodulation modalities such as Deep-Brain Stimulation (DBS).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows ultrasound-transducer targeting of the STN and the GPi to test the feasibility of using DBS for treatment of Parkinson's Disease, in accordance with embodiments;

FIG. 2 shows targeting of the Cingulate Genu to test the feasibility of using DBS for the treatment of Depression, in accordance with embodiments;

FIG. 3 demonstrates ultrasound neuromodulation of the spinal cord to test the feasibility of using Spinal-Cord Stimulation (SCS) for the treatment of neuropathic or ischemic pain, in accordance with embodiments;

FIGS. 4A and 4B show the mechanism for mechanical perturbation and examples the resultant ultrasound field shapes, in accordance with embodiments;

FIG. 5 shows a block diagram of the control circuit, in accordance with embodiments;

FIG. 6 shows a block diagram of feedback control circuit, in accordance with embodiments;

FIG. 7 illustrates a method and steps for pre-planning, in accordance with embodiments;

FIG. 8 illustrates a method and steps for diagnosis, in accordance with embodiments; and

FIG. 9 shows an apparatus to one or more of diagnose or treat the patient, in accordance with embodiments.

### DETAILED DESCRIPTION OF THE INVENTION

The embodiments as described herein provide methods and systems for non-invasive neuromodulation using ultrasound to one or more of diagnosis or to evaluate the feasibility of and preplan neuromodulation treatment using other modalities, such as drugs, electrical stimulation, transcranial ultrasound neuromodulation, surgical intervention, transcranial direct current stimulation, optogenetics, implantable devices, or implantable electrodes and combinations thereof, for example.

In many embodiments, the patient can be diagnosed by selecting one or more target sites. The one or more sites are provided with the focused ultrasound beam. An evaluation of the elicited response to the ultrasound beam may be used to distinguish between one or more patient disorders. The patient treatment can be guided by the disorder identified. The guided treatment may comprise one or more of drugs, neuromodulation, or surgery, for example.

In many embodiments confirming a treatment site encompasses determining which of one or more target neural sites can effectively treat the symptoms to be mitigated, based on identification of the one or more target sites from among a plurality of possible target sites based on a response of the patient to the focused ultrasound beam applied to one or more of the possible target sites.

In many embodiments, the confirmed target site is treated with the non-ultrasonic treatment modality after the confirmed target has been determined to be effective based on the patient's response to focused ultrasonic beam delivered to the target site. In many embodiments, the confirmed target site comprises a target site determined to be most likely to successfully treat the patient. The confirmed target site can be selected from among a plurality of possible target sites evaluated based on the response of the patient to the focused ultrasonic beam.

In many embodiments, the confirmation that treatment at a specific site is effective based on ultrasound occurs before implanting the electrode or other implantable device, for example.

The confirmation of the target site allows one to determine which neural target or targets among a plurality of potential targets will most effectively deal with the symptoms to be mitigated. Such neuromodulation systems can produce applicable acute or long-term effects. The long-term effects can occur through Long-Term Depression (LTD) or Long-Term Potentiation (LTP) via training, for example. The embodiments described herein provide control of direction of the energy emission, intensity, frequency (carrier frequency and/or neuromodulation frequency), pulse duration, pulse pattern, and phase/intensity relationships to targeting and accomplishing up-regulation and/or down-regulation, for example.

In some embodiments, the stimulation frequency for inhibition may be lower than 500 Hz (depending on condition and patient). In an embodiment of the invention, the stimulation frequency for excitation is in the range of 500 Hz to 5 MHz. In an embodiment, the ultrasound acoustic carrier frequency is in range of 0.3 MHz to 0.8 MHz with power generally applied less than 60 mW/cm2 but also at higher target- or patient-specific levels at which no tissue damage is caused. In other embodiments, the ultrasound acoustic carrier frequency can be in range of 0.1 MHz to 0.3 MHz. Alternatively or in combination, the ultrasound acoustic carrier frequency can be in range of 0.8 MHz to 10 MHz, for example. The stimulation frequency can be provided by modulating the ultrasound acoustic carrier frequency with the stimulation frequency, for example.

In many embodiments, the lower limit of the spatial-peak temporal-average intensity (Iₛₚₜₐ) of the ultrasound energy at a target tissue site is chosen from the group of: 21 mW/cm², 25 mW/cm², 30 mW/cm², 40 mW/cm², or 50 mW/cm², for example. In an embodiment of the invention, the upper limit of the Iₛₚₜₐ of the ultrasound energy at a target tissue site is chosen from the group of: 1000 mW/cm², 500 mW/cm², 300 mW/cm², 200 mW/cm², 100 mW/cm², 75 mW/cm², or 50 mW/cm².

In an embodiment of the invention, the acoustic frequency is modulated so as to impact the neuronal structures as desired (e.g., say typically 300 Hz for inhibition (down-regulation) or 1 kHz for excitation (up-regulation), for example).

In many embodiments, the modulation frequency may be divided into pulses 0.1 to 20 msec, and the modulation frequency may be superimposed on the ultrasound carrier frequency, which can be about 0.5 MHz, for example.

In an embodiment, the pulses are repeated at frequencies of 2 Hz or lower for down regulation and higher than 2 Hz for up regulation although this will be both patient and condition specific.

The number of ultrasound transducers can vary between one and five hundred, for example.

In many embodiment, ultrasound therapy is combined with therapy using other neuromodulation modalities, such as one or more of Transcranial Magnetic Stimulation (TMS) or transcranial Direct Current Stimulation (tDCS), for example.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a known commercially available 1-inch diameter ultrasound transducer and a focal length of 2 inches that will deliver a focused spot with a diameter (6 dB) of 0.29 inches with 0.4 MHz excitation. In many embodiments, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.” Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

The ultrasound neuromodulation can be administered in sessions. Examples of session types include periodic sessions, such as a single session of length in the range from 15 to 60 minutes repeated daily or five days per week for one to six weeks. Other lengths of session or number of weeks of neuromodulation are applicable, such as session lengths from 1 minute up to 2.5 hours and number of weeks ranging from one to eight. Sessions occurring in a compressed time period typically means a single session of length in the range from 30 to 60 minutes repeated during with inter-session times of 15 minutes to 60 minutes over one to three days. Other inter-session times in the range between 1 minute and three hours and days of compressed therapy such as one to five days are applicable. In an embodiment of the invention, sessions occur only during waking hours. Maintenance consists of periodic sessions at fixed intervals or on as-needed basis such as occurs periodically for tune-ups. Maintenance categories are maintenance post-completion of original treatment at fixed intervals and maintenance post-completion of original treatment with as-needed maintenance tune-ups as defined by a clinically relevant measurement. In an embodiment that uses fixed intervals to determine when additional ultrasound neuromodulation sessions are delivered, one or more 50-minute sessions occur during the second week the 4^{th} and 8^{th} months following the first treatment. In an embodiment that when additional ultrasound neuromodulation sessions are delivered based on a clinically-relevant measurement, one or more 50-minute sessions occur during week 7 because a tune up is needed at that time as indicated by the re-emergence of symptoms. Use of sessions is important for the retraining of neural pathways for change of function, maintenance of function, or restoration of function. Retraining over time, with intermittent reinforcement, can more effectively achieve desired impacts. Efficient schedules for sessions are advantageous so that patients can minimize the amount of time required for their ultrasound treatments. Such neuromodulation systems can produce applicable acute or long-term effects. The latter occur through Long-Term Depression (LTD) or Long-Term Potentiation (LTP) via training

Work in relation to embodiments as described herein suggests that differences in FUP phase, frequency, and amplitude produce different neural effects. Low frequencies (defined as below 500 Hz.) can be inhibitory in at least some embodiments. High frequencies (defined as being in the range of 500 Hz to 5 MHz) can be excitatory and activate neural circuits in at least some embodiments. In many embodiments, this targeted inhibition or excitation based on frequency works for the targeted region comprising one or more of gray or white matter. Repeated sessions may result in long-term effects. The cap and transducers to be employed can be preferably made of non-ferrous material to reduce image distortion in fMRI imaging, for example. In many embodiments, if after treatment the reactivity as judged with fMRI of the patient with a given condition becomes more like that of a normal patient, this clinical assessment may be indicative of treatment effectiveness. In many embodiments, the FUP is to be applied 1 ms to 1 s before or after the imaging. Alternatively or in combination, a CT (Computed Tomography) scan can be run to gauge the bone density and structure of the skull, which can be used to determine one or more of the carrier wave frequency, the pulse intensity, the pulse energy, the pulse duration, the pulse repetition rate, or the pulse phase, for a series of pulses as described herein, for example.

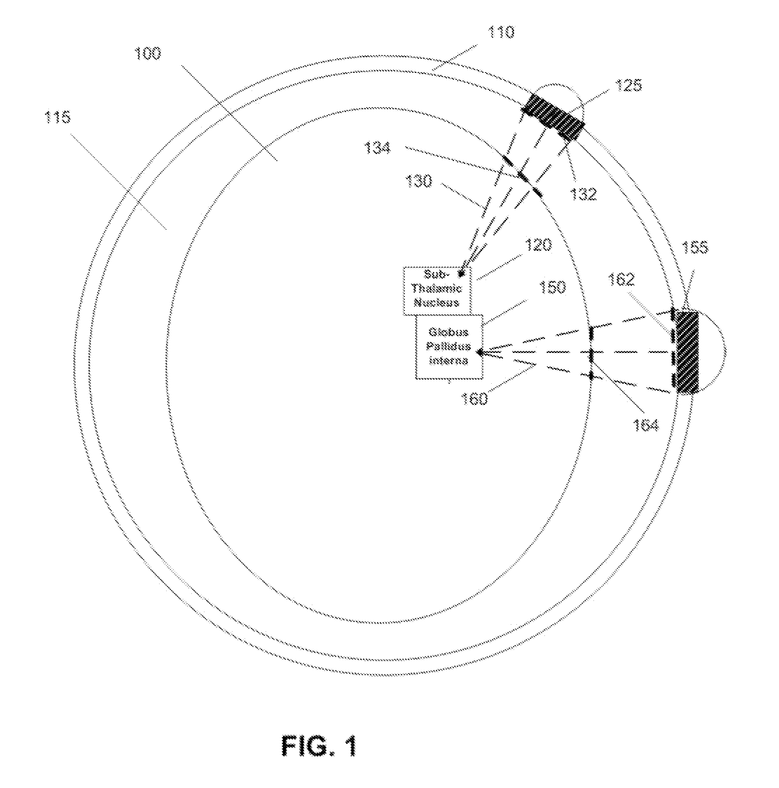

FIG. 1 shows a set of ultrasound transducers targeted to treat Parkinson's Disease. Head 100 contains two targets, Subthalamic Nucleus 120 and Globus Pallidus internal 150. The targets shown are hit by ultrasound from transducers 125 and 155 fixed to track 110. Ultrasound transducer 125 with its beam 130 is shown targeting Subthalamic Nucleus (STN) 120 and transducer 155 with its beam 160 is shown targeting Globus Pallidus internal 150. For ultrasound to be effectively transmitted to and through the skull and to brain targets, coupling must be put into place. Ultrasound transmission (for example Dermasol from California Medical Innovations) medium 115 is interposed with one mechanical interface to the frame 110 and ultrasound transducers 125 and 155 (completed by a layers of ultrasound transmission gel 132 and 162 respectively) and the other mechanical interface to the head 100 (completed by a layers of ultrasound transmission gel 134 and 164 respectively). In another embodiment the ultrasound transmission gel is placed around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers. In still another embodiment, an alternative target can be evaluated with ultrasound neuromodulation, such the Vim (Ventral Intermediate Nucleus of the Thalamus). A diagnostic application of the invention is the differentiation between the tremor of Parkinson's Disease and essential tremor. Note that one strategy is to use DBS on both the STN and the Vim on the same side. In another embodiment, ultrasound neuromodulation of the spinal cord is used to evaluate the potential effectiveness of or parameters for Spinal Cord Stimulation (SCS) using invasive electrode stimulation for the relief of pain.

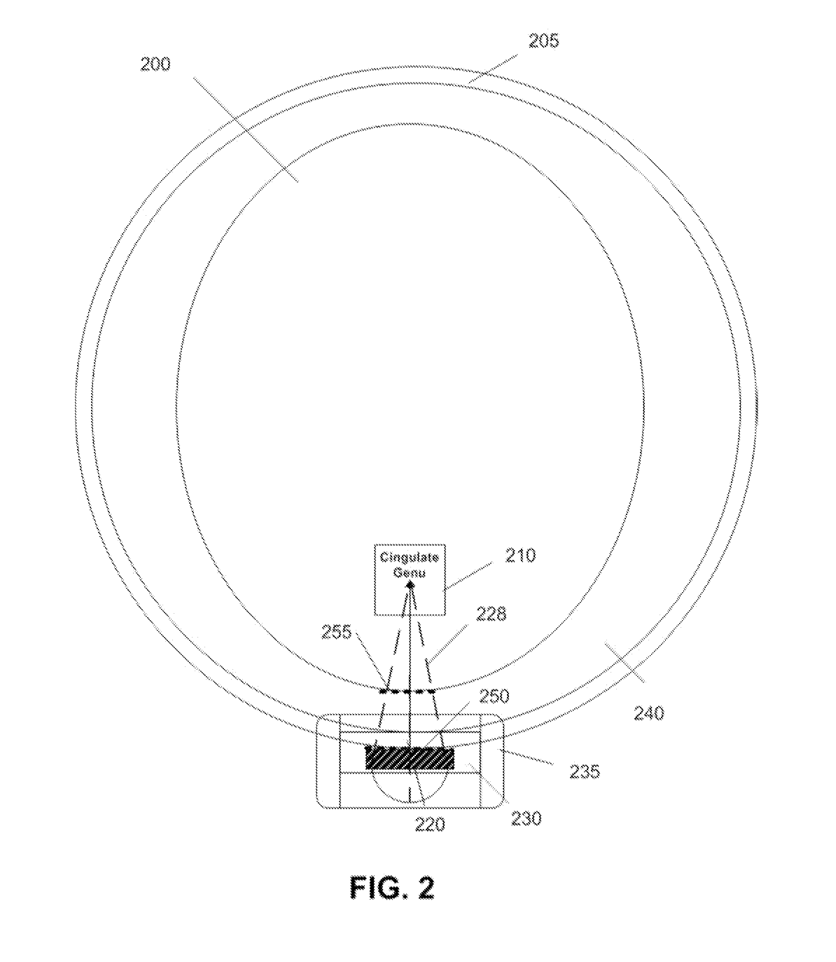

FIG. 2 illustrates the Cingulate Genu as a target for testing in a neuromodulation patient to evaluate whether neuromodulation of that target is effective for the mitigation of depression or bipolar disorder. Head 200 is surrounded by head frame 205 on which ultrasound neuromodulation transducer frame 235 containing an adjustment support 230 which moves radially in and out of transducer frame 235. Support 230 holds ultrasound transducer 220 with its ultrasound beam 228 hitting target being evaluated Cingulate Genu 210. In order for the ultrasound beam 228 to penetrate effectively, an ultrasound conduction path must be used. This path consists of ultrasound conduction medium 240 (for example Dermasol from California Medical Innovations) bounded by ultrasound conduction-gel layer 250 on the ultrasound-transducer side and layer 255 on the head side. If the ultrasound neuromodulation is successful, then an alternative neuromodulation modality (e.g., DBS) likely can be used successfully due to smaller targeting area achieved. If the ultrasound neuromodulation of this target is not effective then it is likely that the alternative modality being considered (e.g., DBS) will not be successful with this target. Thus the probability of success with an alternative (potentially invasive) neurmodulation modality can be evaluated. If an acute session of ultrasound neuromodulation is ineffective for alleviating symptoms, then the probability is lower that the patient will benefit from a more invasive procedure such as invasive DBS, avoiding both risk for side effects in the patient and significant cost.

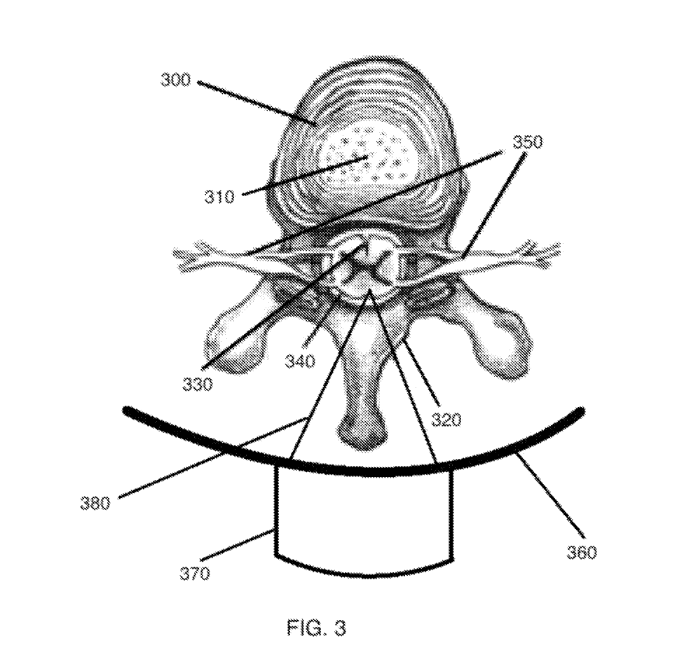

FIG. 3 shows a cross section of the spinal column and spinal cord. Applying ultrasound neuromodulation in this configuration is useful for preplanning to evaluate whether electrode-based Spinal Cord Stimulation (SCS) would be effective in a patient and how SCS should be targeted. Vertebrae disc 300 including nucleus pulposus 310 and other bony structures such as the lamina 320 covers the dura 340 that surrounds the spinal cord 330 with its spinal nerve roots 350. Ultrasound transducer 370 is pressed against skin 360 and generates ultrasound beam 380 that neuromodulates nerves within spinal cord 330. Bilateral neuromodulation of spinal cord 330 can be performed. For ultrasound to be effectively transmitted to and through the skin and to target spinal-cord target, coupling must be put into place. A layer of ultrasound transmission gel (not shown) is placed between the face of the ultrasound transducer and the skin over the target. If filling of additional space (e.g., within the transducer housing) is necessary, an ultrasound transmission medium (for example Dermasol from California Medical Innovations) can be used. In another embodiment, multiple ultrasound transducers whose beams intersect at that target replace an individual ultrasound transducer for that target. In still another embodiment, mechanical perturbations are applied radially or axially to move the ultrasound transducers. Ultrasound neuromodulation locations that are successful suggest sites at which application of Spinal Cord Stimulation is likely to also be successful. In an embodiment of the invention, effective parameters of the ultrasound neuromodulation can provide insight into the parameters to be used in SCS, for instance pulsing frequency, relative intensity, and whether a stimulus is monophasic or biphasic.

Transducer array assemblies of the type used in ultrasound neuromodulation may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—31 July-2 Aug. 2002), typically with numbers of ultrasound transducers of 300 or more. Keramos-Etalon and Blatek in the U.S. are other custom-transducer suppliers. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

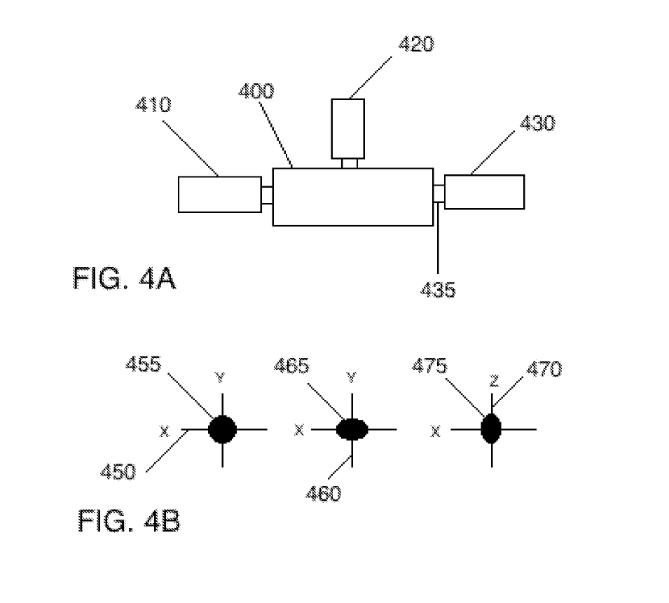

FIGS. 4A and 4B show the mechanism for mechanical perturbation of the ultrasound transducer. In FIG. 4A illustrates a plan view with mechanical actuators 420 and 430 moving ultrasound transducer 400 in and out and left respectively. Actuator rod 435 provides the mechanical interface between mechanical actuator 430 and ultrasound transducer 400 as an example. An equivalent mechanical actuator 410 is shown schematically and moves ultrasound transducer 400 along an axis perpendicular to the page. The combination of actuator 410, actuator 420 and actuator 430 can provide three-dimensional scan patterns under control of the system and under user input as described herein. Such mechanical actuators can have alternative configurations such as motors, vibrators, solenoids, magnetostrictive, electrorestrictive ceramic and shape memory alloys. Piezo-actuators such as those provided by DSM can have very fine motions of 0.1% length change. FIG. 4B shows effects on the focused ultrasound modulation focused at the target. The three axes are axis 450 (x,y), axis 460 (x,y,) and axis 470 (x,z). As demonstrated on the axes 450 the excursions along x and y from actuator 430 and actuator 420, respectively, are equal so the resultant pattern is a circle. As demonstrated on axis 460 the excursion due to actuator 430 is greater than that actuator 420 so the resultant pattern is longer along the x axis than the y axis. As demonstrated on axis 470, the excursion is longer along the z axis than the x axis to the resultant pattern is long along the z axis than the x axis. Not shown is the inclusion of the impacts of actuation along the axis perpendicular to the page, although this will be readily understood by a person of ordinary skill in the art. In each case, the pattern of movement can be determined so as to correspond to the shape of the target site treated with the modulated ultrasound beam.

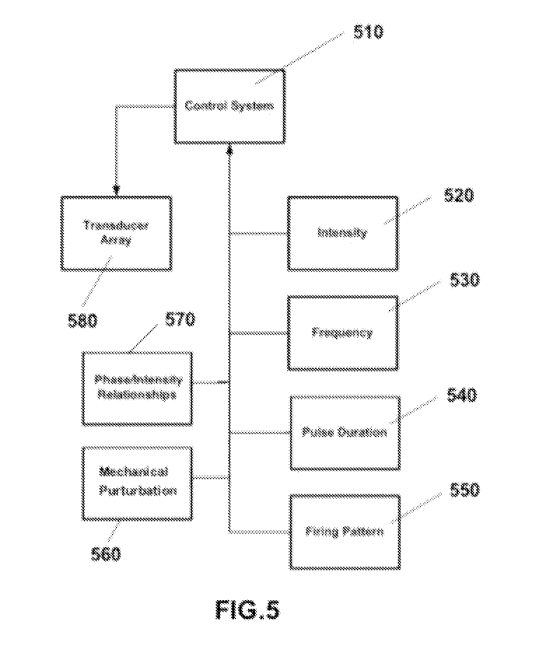

FIG. 5 shows an embodiment of a control circuit. The positioning and emission characteristics of transducer array 580 are controlled by control system 510 with control input with neuromodulation characteristics determined by settings of intensity 520, frequency 530, pulse duration 540, firing pattern 550, mechanical perturbation 560, and phase/intensity relationships 570 for beam steering and focusing on neural targets.

The patient can be treated in one or more of many ways. For example, the patient can be treated with one or more sessions. The pulse can be shaped in many ways with one or more of macro pulse shaping and amplitude modulation, for example. For example, the ultrasound acoustic carrier frequency can be pulse shape modulated, so as to provide shaped stimulation pulses comprising ultrasound having the carrier frequency.

In another embodiment, a feedback mechanism to ultrasound stimulation is applied such as functional Magnetic Resonance Imaging (fMRI), Positive Emission Tomography (PET) imaging, video-electroencephalogram (V-EEG), acoustic monitoring, thermal monitoring, and patient feedback. In an embodiment, feedback is provided by a measurement specific to a symptom or disease state of a patient.

In still other embodiments, other energy sources are used in combination with or substituted for ultrasound transducers such as Transcranial Magnetic Stimulation (TMS) or transcranial Direct Current Stimulation (tDCS). Therapies that can be preplanned with ultrasound neuromodulation are usually invasive modalities such as Deep-Brain Stimulation (DBS), optogenetics application, or stereotactic radiosurgery. Alternatively ultrasound neuromodulation can be used for preplanning for non-invasive neuromodulation such as Transcranial Magnetic Stimulation (TMS) or transcranial Direct Current Stimulation (tDCS). In either or both cases preplanning can be done for one or multiple modalities including the aforementioned and other therapies such as behavioral therapies and drugs.

The operator can set the variables for preplanning or diagnostic ultrasound neuromodulation or the patient can do so in a self-actuated manner. In some self-actuated embodiments, the patient can expedite the process due to their ability to tune the ultrasound neuromodulation to obtain its best results through subjective assessments of whether a symptom or disease state is mitigated with a particular ultrasound session.

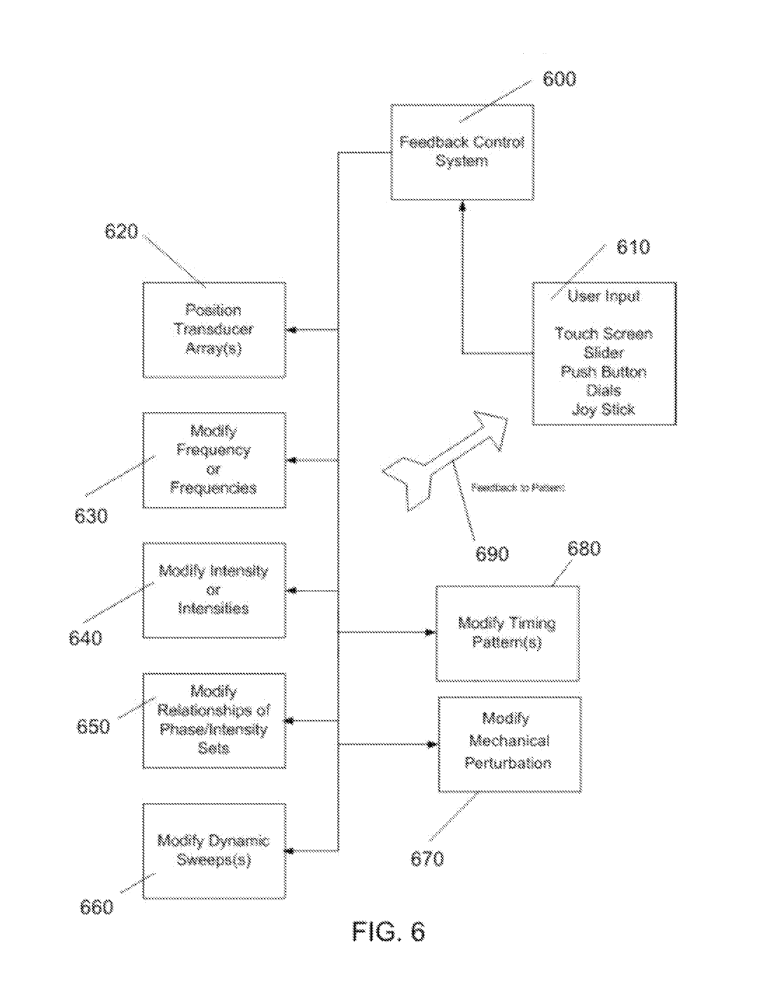

FIG. 6 shows the basic feedback circuit. Feedback Control System 600 receives its input from User Input 610 and provides control output for positioning ultrasound transducer arrays 620, modifying pulse frequency or frequencies 630, modifying intensity or intensities 640, modifying relationships of phase/intensity sets 650 for focusing including spot positioning via beam steering, modifying dynamic sweep patterns 660, modifying mechanical perturbation 670, and/or modifying timing patterns 680. Feedback to the patient 690 occurs based on a measured physiological cognitive, subjective, or other disease-or health-related measurement (for example increase or decrease in pain or decrease or increase on tremor). User Input 520 can be provided via a touch screen, slider, dials, joystick, or other suitable means. Often the user can be the best judge concerning which neuromodulation parameters are most effective, either changing one variable of ultrasound at a time or multiple ultrasound waveform variables. Examples of the application of patient feedback are the patient adjusting neuromodulation parameters to ameliorate pain, depression, and resting tremor. Another is a patient with a transected spinal cord directly turning on the neuromodulation to empty a neurogenic bladder.

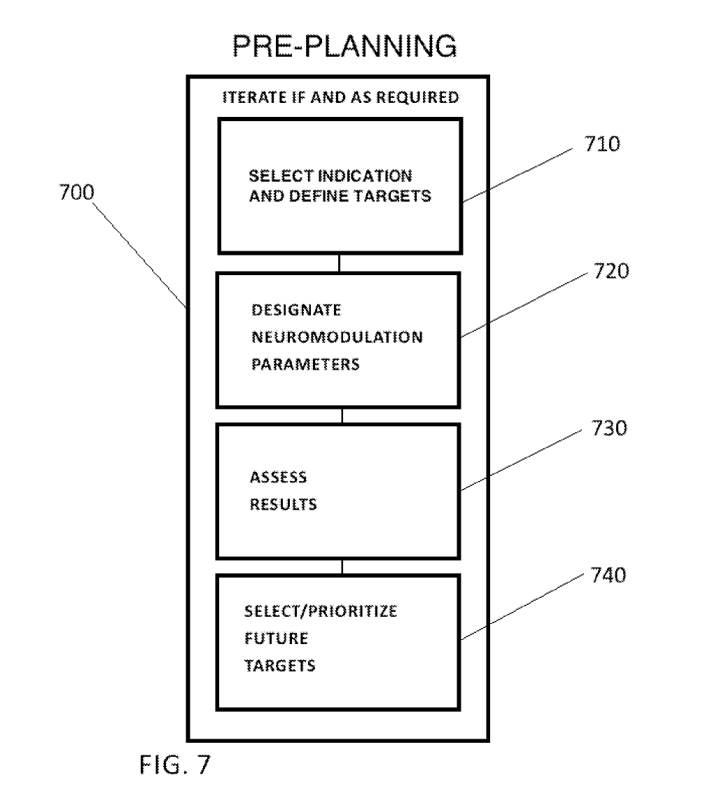

FIG. 7 shows a method 700 of pre-planning for neuromodulation therapy. The neuromodulation therapy may comprise one or more of Ultrasound Neuromodulation, Transcranial Magnetic Stimulation (TMS) or Deep Brain Stimulation (DBS)) or ablative therapy, for example. Each of the steps within method 700 may be performed iteratively, for example. A step 710 comprises selecting an indication for treatment and defining related targets sites. The indication may comprise one or more indications as described herein such as one or more of Parkinson's Disease, Depression/Bipolar Disorder, or Spinal Cord Pain, for example. A step 720 comprises designating ultrasound neuromodulation parameters to apply in either one or multiple neuromodulation sessions, for example. The neuromodulation parameters may comprise one or more known parameters and can be determined by one of ordinary skill in the art based on the embodiments described herein. A step 730 comprises assessing the results in response to the ultrasound neuromodulation in order to determine stimulation effect, if present. The presence of a stimulation effect can confirm the site as suitable for use with treatment. A step 740 comprises one or more of selecting or prioritizing targets for future treatment based on the assessment of the results, such that the sites are confirmed prior to treatment.

Table 1 shows a table suitable for incorporation with pre-planning in accordance with embodiments as described herein.

TABLE 1

| Condition- Input | Target Site Evaluated | Assessment | Subsequent Treatment |
|---|---|---|---|
| Depression | Cingulate Genu | Depression/Normal | DBS targeted to |
| | | | cingulate genu |
| Parkinson's | DBS, STN, GPi | Tremor | levodopa, dopamine |
| | | | agonists, MAO-B |
| | | | inhibitors, and other |
| | | | drugs such as |
| | | | amantadine and |
| | | | anticholinergics |
| Essential Tremor | (Vim) | Tremor | beta blockers, |
| | | | propranolol, |
| | | | antiepileptic agents, |
| | | | primidone, or |
| | | | gabapentin |
| Bipolar Disorder | Nucleus accumbens, | Structured Clinical | DBS, lithium, valproic |
| | the subcallosal | Interview for DSM-IV | acid, divalproex, |
| | cingulate (Area 25) | (SCID), the Schedule | lamotrigine, |
| | | for Affective Disorders | quetiapine, |
| | | and Schizophrenia | antidepressants, |
| | | (SADS), or other | Symbyax, clonazepam, |
| | | bipolar assessment tool | lorazepam, diazepam, |
| | | | chlordiazepoxide, and |
| | | | alprazolam |
| Spinal Cord Pain | Various levels of the | Comparative pain scale | Level of the spinal |
| | spinal column; white | or galvanic skin | column and site for |
| | matter and ganglia | response | electrical stimulation, |
| | | | ultrasound |
| | | | neuromodulation, or |
| | | | surgical intervention |

With regards to the Nucleus accumbens, supportive data can be found be one of ordinary skill in the art on the world wide web (www.clinicaltrials.gov/ct2/show/NCT01372722). With regards to the subcallosal cingulate (Area 25), supportive data can be found be one of ordinary skill in the art on the world wide web (www.dana.org/media/detail.aspx?id=35782). With regards to the Schedule of Affective Disorders and Schizophrenia, supportive data can be found by one of ordinary skill in the art at on the world wide web (www.ncbi.nlm.nih.gov/pmc/articles/PMC2847794/). With regards to treatment and drugs related to bipolar disorder, supportive data can be found on the world wide web by one of ordinary skill in the art (http://www.mayoclinic.com/health/bipolar-disorder/DS00356/DSECTION=treatments-and-drugs).

The method 700 can be used to confirm treatment of the patient based on the patient's response to target site evaluated. For the condition input and target site evaluated, a subsequent treatment can be selected that acts on the target site evaluated, for example as described herein with reference to Table 1.

Although the above steps show method 700 of planning a treatment of a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the treatment.

One or more of the steps of the method 700 may be performed with the circuitry as described herein, for example one or more of the processor or logic circuitry such as programmable array logic for field programmable gate array. The circuitry may be programmed to provide one or more of the steps of method 700, and the program may comprise program instructions stored on a computer readable memory or programmed steps of the logic circuitry such as the programmable array logic or the field programmable gate array, for example.

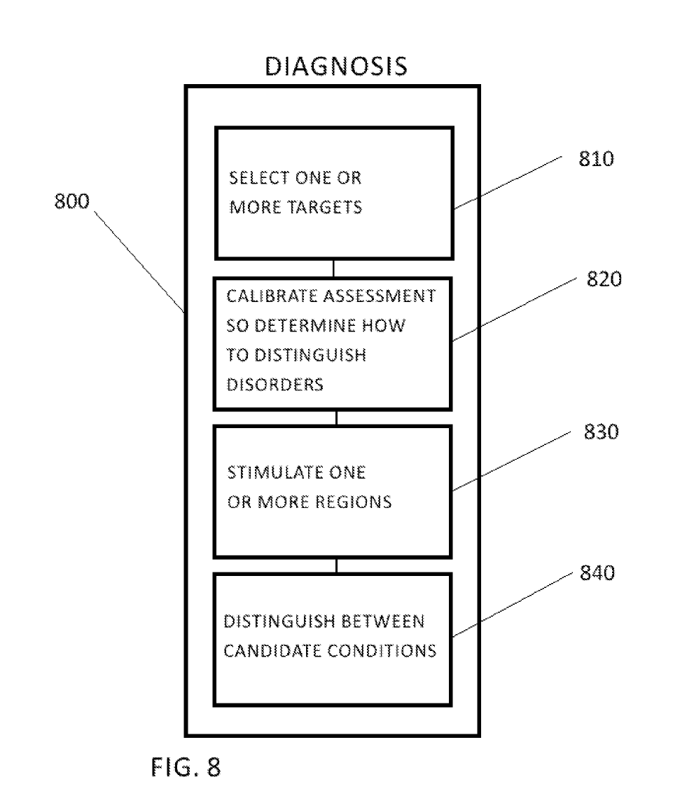

FIG. 8 shows a method 800 of diagnosis of a patient. A step 810 comprises selection of one or more target sites as described herein. A step 820 comprises calibrating an assessment to determine how to distinguish candidate disorders based on elicited effects consistent with one disorder versus another disorder, for example. A step 830 comprises stimulating the one or more target sites with ultrasound as described herein. A step 840 comprises distinguishing among a plurality of candidate conditions. The process 800 provides information for guiding treatment irrespective of the treatment. The treatment may comprise one or more treatments as described herein such as neuromodulation, surgery, or medication, for example. Assessments can be made by direct observation or by instruments such as the known Visual Analog Scale for pain (H. Breivik, H., Borchgrevink, P. C., Allen, S. M., Rosseland, L. A., Romundstad, L., Breivik Hals, E. K., Kvarstein, G., and A. Stubhaug, “Assessment of Pain,” Br J Anaesth. 2008; 101(1):17-24.) or motor skill assessments for Parkinson's disease (Motor Bruininks-Oseretsky Test of Motor Proficiency, Second Edition (BOT-2), Authors: Robert H. Bruininks, PhD & Brett D. Bruininks, (for ages for four through 21) and Bruininks Motor Ability Test (BMAT), Authors: Brett D. Bruininks & Robert H. Bruininks, PhD (for adults), both by Pearson Education, Inc.).

Table 2 shows a table suitable for incorporation with diagnosis in accordance with embodiments as described herein.

TABLE 2

| | Target Site(s) | | |
|---|---|---|---|
| Symptom- Input | Evaluated- Input | Assessment/Indicator | Condition- Output |
| Depression/Normal | Cingulate Genu | Depression/Normal | Depression |
| Tremor | DBS, STN, or GPi | Tremor | Parkinson's |
| Tremor | Vim | Tremor | Essential Tremor |
| Bipolar behavior | Nucleus accumbens, | Structured Clinical | Bipolar Disorder |
| | the subcallosal | Interview for DSM-IV | |
| | cingulate (Area 25) | (SCID), the Schedule | |
| | | for Affective Disorders | |
| | | and Schizophrenia | |
| | | (SADS), or other | |
| | | bipolar assessment tool | |
| Pain | Spinal Cord; Various | Comparative pain scale | Spinal Cord Pain |
| | levels of the spinal | or galvanic skin | |
| | column; white matter | response | |
| | and ganglia | | |

Although the above steps show method 800 of diagnosing a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the treatment.

One or more of the steps of the method 800 may be performed with the circuitry as described herein, for example one or more of the processor or logic circuitry such as programmable array logic for field programmable gate array. The circuitry may be programmed to provide one or more of the steps of method 800, and the program may comprise program instructions stored on a computer readable memory or programmed steps of the logic circuitry such as the programmable array logic or the field programmable gate array, for example.

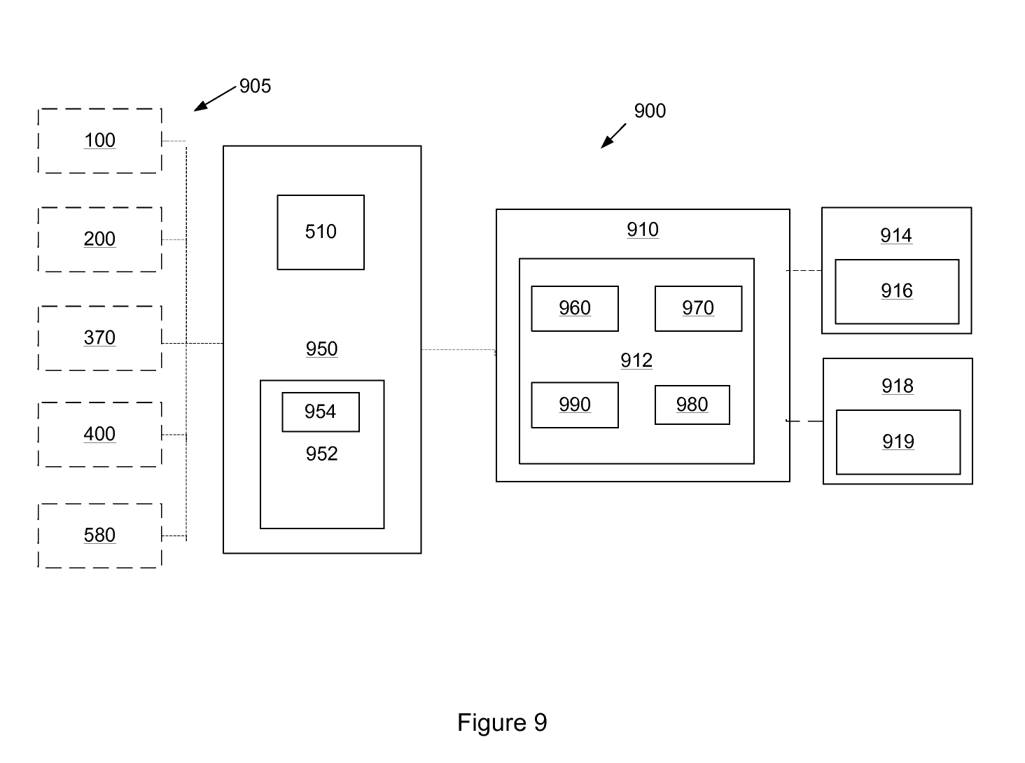

FIG. 9 shows an apparatus 900 for one or more of preplanning or diagnosing the patient, in accordance with embodiments. The apparatus 900 comprises an ultrasound source 905. The ultrasound source 905 comprises a source of ultrasound as described herein. The ultrasound source 905 may comprise a head 100, a head 200, a transducer 370, a transducer 400, or a transducer array 580 as described herein for example.

The apparatus 900 comprises a controller 950 coupled to the ultrasound source 905. The controller 950 comprises a processer 952 having a computer readable medium 954. The computer readable memory 954 may comprise instructions for controlling the ultrasound source. The controller 950 may comprise one or more components of the control system 510 as described herein.

The apparatus 900 comprises a processor system 910. The processor system 910 is coupled with a control system. The processor 910 comprises a computer readable memory 912 having instructions of one or more computer programs embodied thereon. The computer readable memory 912 comprises instructions 960. The instructions 960 comprise one or more instructions of the feedback control system 600 and corresponding methods as described herein. The computer readable memory 912 comprises instructions 970. The instructions 970 comprise one or more instructions to implement one or more steps of the preplanning method 700 as described herein. The computer readable memory 980 comprises instructions to implement one or more steps of the method 980 of diagnosing a patient as described herein. The computer readable memory 912 comprises instructions 990 to coordinate the components as described herein and the methods as described herein. For example, the instructions 990 may comprise a user responsive switch to select preplanning method 970 or instructions to diagnose the patient 980 based on user preference. The computer readable memory may comprise information of one or more of Table 1 or Table 2 so as to plan treatment of the patient and diagnose the patient, in accordance with embodiments as described herein.

The processor system 910 is coupled to a user interface 914. The user interface 914 may comprise a display 916 such as a touch screen display. The user interface 914 may comprise a handheld device such as a commercially available iPhone, Android operating system device, such as, a Samsung Galaxy S3 or other known handheld device such as an iPad, tablet computer, or the like. The user interface 914 can be coupled with a processor system 910 with communication methods and circuitry. The communication may comprise one or more of many known communication techniques such as WiFi, Bluetooth, cellular data connection, and the like. The processor system 910 is configured to communicate with a measurement apparatus 918. The measurement apparatus 918 comprises patient measurement data storage 919 that can be stored on a computer readable memory. The processor system 910 is in communication with the measurement apparatus 918 with communication that may comprise known communication as described herein. The processor system 910 is configured to communicate with the controller 950 to transmit the signals for use with the ultrasound source 905 in for implementation with one or more components of control system 510 as described herein.

The apparatus 900 allows ultrasound stimulation adjustments in variables such as carrier frequency and/or neuromodulation frequency, pulse duration, pulse pattern, mechanical perturbation, as well as the direction of the energy emission, intensity, frequency, phase/intensity relationships to targeting and accomplishing up-regulation and/or down-regulation, dynamic sweeps, and position. The user can input these parameters with the user interface, for example.

Reference is made to the following publications, which are provided herein to clearly and further show that the embodiments of the methods and apparatus as described herein are clearly enabled and can be practiced by a person of ordinary skill in the art without undue experimentation.

Clinical stimulation of the Cingulate Genu in humans is described by Mayberg et al. (Mayberg, Helen S., Lozano, A. M., Voon, Valerie, McNeely, Heather E., Seminowicz, D., Hamani, C., Schwalb, J. M., and S. H., Kennedy, “Deep Brain Stimulation for Treatment-Resistant Depression,” Neuron, Volume 45, Issue 5, 3 Mar. 2005, Pages 651-660), for example.

Patient response to Stimulation of the Subthalamic Nucleus and Globus Pallidus interna can produce measurable patient results suitable for one or more of diagnosis or confirmation as described herein. (Anderson et al. (Anderson, V C, Burchiel, K J, Hogarth, P, Favre, J, and J P Hammerstad, “Pallidal vs subthalamic nucleus deep brain stimulation in Parkinson disease,” Arch Neurol. 2005 April; 62(4):554-60)

The stimulation of deep-brain structures with ultrasound has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton describes a strong DC magnetic field as well and describes the mechanism as that given that the tissue to be stimulated is conductive that particle motion induced by an ultrasonic wave will induce an electric current density generated by Lorentz forces, such that ultrasound is suitable for combination with TMS in accordance with embodiments as described herein.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as compared to TMS focused to no more than 1 cm. However, a person of ordinary skill in the art can combine ultrasound with TMS in accordance with the embodiments as described herein.

Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output, suitable for combination in accordance with embodiments as described herein. Transducers may coordinated by a computer and used in conjunction with an imaging system, preferable an fMRI (functional Magnetic Resonance Imaging), but possibly a PET (Positron Emission Tomography) or V-EEG (Video-Electroencephalography) device. The user may interact with the computer to direct the FUP to the desired point in the brain, sees where the stimulation actually occurred by viewing the imaging result, and thus adjusts the position of the FUP accordingly.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of neuromodulation of a patient, the method comprising:
providing pulsed ultrasound to one or more neural targets of a neural disorder; and
identifying the neural disorder or planning for treatment of the neural disorder based on a response of the one or more neural targets to the pulsed ultrasound.

2. The method of claim 1 wherein planning for treatment of the neural disorder comprises determining parameters of the pulsed ultrasound in order to confirm a neuromodulation therapy in order to treat the neural disorder based on a response of the one or more neural targets to the parameters.

3. The method of claim 1 wherein planning for treatment comprises preplanning for a neuromodulation therapy comprising one or more of surgical, invasive neuromodulation, non-invasive neuromodulation, behavioral therapy, or drugs.

4. The method of claim 1 wherein patient feedback is used to adjust symptoms selected from the group of pain, depression, tremor, voiding from neurogenic bladder; and wherein the symptoms are adjusted based on the one or more neural targets and parameters of the pulsed ultrasound.

5. The method of claim 1 wherein the identifying the neural disorder comprising differentiating between the tremor of Parkinson's Disease and essential tremor.

6. The method of claim 1 wherein the planning for treatment comprises identifying a response to neuromodulation of the Cingulate Genu for the purpose of treating depression.

7. The method of claim 1 wherein planning for treatment comprises identifying a response to neuromodulation of the spinal cord for the purpose of reducing pain.

8. The method of claim 1 wherein the one or more targets are neuromodulated in a manner selected from the group consisting of ipsilateral neurmodulation, contralateral neuromodulation, and bilateral neuromodulation.

9. The method of claim 1 wherein one or more energy sources is used to treat the neural disorder, the one or more energy sources selected from the group consisting of Transcranial Magnetic Stimulation (TMS) and transcranial Direct Current Stimulation (tDCS).

10. The method of claim 1 wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, and a subjective patient response.

11. A system for neuromodulation, the system comprising:
circuitry coupled to one or more ultrasound transducers to provide pulsed ultrasound to one or more neural targets;
a processor coupled to the circuitry, the processor configured to identify a neural disorder or plan for treatment of the neural disorder based on a response of the one or more neural targets to the pulsed ultrasound.

12. The system of claim 11 wherein the processor comprises instructions to plan for treatment of the neural disorder, including determining parameters of the pulsed ultrasound in order to confirm a neuromodulation therapy in order to treat the neural disorder based on a response of the one or more neural targets to the parameters.

13. The system of claim 11 wherein the processor comprises instructions to plan for treatment, including preplanning for a neuromodulation therapy comprising one or more of surgical, invasive neuromodulation, non-invasive neuromodulation, behavioral therapy, or drugs.

14. The system of claim 11 wherein the processor comprises instructions to receive patient feedback in order to adjust symptoms selected from the group of pain, depression, tremor, voiding from neurogenic bladder; and wherein the symptoms are adjusted based on the one or more neural targets and parameters of the pulsed ultrasound.

15. The system of claim 11 wherein the processor comprises instructions to identify the neural disorder comprising differentiating between the tremor of Parkinson's Disease and essential tremor.

16. The system of claim 11 wherein the processor comprises instructions to plan for treatment, including identifying a response to neuromodulation of the Cingulate Genu for the purpose of treating depression.

17. The system of claim 11 wherein the processor comprises instructions to plan for treatment, including identifying a response to neuromodulation of the spinal cord for the purpose of reducing pain.

18. The system of claim 11 wherein the processor comprises instructions to neuromodulate the one or more targets in a manner selected from the group consisting of ipsilateral neurmodulation, contralateral neuromodulation, and bilateral neuromodulation.

19. The system of claim 11 wherein the processor comprises instruction to preplan for treatment based on one or more energy sources which is used to treat the neural disorder, the one or more energy sources selected from the group consisting of Transcranial Magnetic Stimulation (TMS) and transcranial Direct Current Stimulation (tDCS).

20. The system of claim 11 wherein the processor system comprises instructions of an applied feedback mechanism, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, and a subjective patient response.

21. The system of claim 11 wherein the processor system comprises instructions to pre-plan for treatment of the neural disorder and wherein the neural disorder comprises one or more of depression, Parkinson's disease, essential tremor, bipolar disorder or spinal cord pain and wherein the target site evaluated prior to treatment comprises one or more of a Cingulate Genu, DBS, STN, GPi, Vim, Nucleus accumbens, Area 25 of subcallosal cingulate, one or more levels of a spinal column, white matter or ganglia.

22. The system of claim 11 wherein the processor system comprises instructions to diagnose the neural disorder and wherein a symptom of the neural disorder comprises one or more of depression, tremor, bipolar behavior or pain and wherein the target site evaluated comprises one or more of Cingulate Genu, DBS, STN, GPi, Vim, Nucleus accumbens, area of 25 of subcallosal cingulate, one or more levels of the spinal column, whiter matter or ganglia.
